(19) United States
(12) Patent Application Publication
(10) Pub. No.: US 2021/0068754 A1
(43) Pub. Date: Mar. 11, 2021

(54) TORSO STRAP

(71) Applicant: Beyond Pulse, LLC, Portland, OR (US)

(72) Inventor: Nya Kamhi, New York, NY (US)

(73) Assignee: Beyond Pulse, LLC, Portland, OR (US)

(21) Appl. No.: 17/017,637

(22) Filed: Sep. 10, 2020

Related U.S. Application Data
(60) Provisional application No. 62/898,990, filed on Sep. 11, 2019.

(51) Int. Cl. A61B 5/00; A61B 5/0205

(52) U.S. Cl. CPC A61B 5/6823; A61B 5/02438; A61B 5/6831; A61B 5/02055

(57) ABSTRACT

Apparatus and methods are disclosed for providing a strap configured to be worn around a torso and situate a circuit offset from other garments worn by the strap user. For example, a torso strap can be provided that has an offset portion or lobe that situates a circuit offset from another portion of the torso strap. In some examples, a torso strap includes a narrower portion adapted to encircle the torso, a wider portion configured to situate a circuit at least partially offset from the narrower portion of the strap, a sensor coupled to a first surface of the strap, and a wire being in communication with the circuit and the sensor. In some examples, a method includes providing such an apparatus and using the apparatus to monitor health characteristics of the user, including pulse, heart rate, temperature, blood oxygen, velocity, displacement, attitude, time, or moisture.

### CROSS-REFERENCE TO RELATED APPLICATIONS

This application claims the benefit of U.S. Provisional Application No. 62/898,990, filed Sep. 11, 2019, which application is incorporated herein by reference in its entirety as if set forth fully herein.

### SUMMARY

Apparatus and methods are disclosed for providing a strap configured to be worn around a torso and configurable to situate a circuit in a manner that will be offset from other garments worn by the strap user. For example, a torso strap can be provided that has an offset portion or lobe that situates a circuit offset from another portion of the torso strap. Thus, if a user is wearing the torso strap underneath another garment such as a sports brassiere, physical interference of the circuit with the other garment can be avoided. Such torso straps may be worn with sports bras having elastic or underwire supports. The circuit mounted to the torso strap can be used to collect health information about the user, for example heart rate or temperature, store such data, and communicate such data to other computer enabled components. The data can be used to analyze the user's health or performance in sports-related activities. Typically, such torso straps are worn around a lower chest region of a user's torso.

In some examples of the disclosed technology, an apparatus is provided including a strap configured to be worn around a torso, the strap including a narrower portion adapted to encircle the torso, a wider portion configured to situate a circuit at least partially offset from the narrower portion of the strap, at least one sensor coupled to a first surface of the strap, and at least one wire being in electrical or optical communication between the circuit and the at least one sensor. In some examples, the narrower portion of the strap defines a first longitudinal axis, and the wider portion comprises a lobe that extends along a second axis, the wider portion being further situated to situate the circuit along the second axis and at least partially offset from the first axis. In some examples, the circuit is completely offset from the second axis. In some examples, the circuit is completely offset from a region formed by the strap along the first axis. In some examples of the disclosed technology, a method includes providing such an apparatus and using the apparatus to monitor at least one health characteristic of the wearer/user. For example, health characteristics such as pulse, heart rate, temperature, blood oxygen, velocity, displacement, attitude, time, or moisture can be monitored using the circuit situated on the strap. In some examples of the disclosed technology, a method of manufacturing an apparatus includes providing a strap configured to be worn around a torso, the strap including: a narrower portion adapted to encircle the torso, and a wider portion including a region to situate a circuit at least partially offset from the narrower portion of the strap. The method further includes attaching a circuit to the region, attaching at least one sensor to or below a first surface of the strap, and connecting at least one wire in electrical communication between the circuit and the at least one sensor.

This Summary is provided to introduce a selection of concepts in a simplified form that are further described below in the Detailed Description. All trademarks used herein remain the property of their respective owners. This Summary is not intended to identify key features or essential features of the claimed subject matter, nor is it intended to be used to limit the scope of the claimed subject matter. The foregoing and other objects, features, and advantages of the disclosed subject matter will become more apparent from the following Detailed Description, which proceeds with reference to the accompanying figures.

### BRIEF DESCRIPTION OF THE DRAWINGS

FIG. 1 illustrates two examples of brassieres that can be worn about a torso, as can be implemented in certain examples of the disclosed technology.

FIG. 2 illustrates an apparatus including a strap configured to be worn around a torso, as can be implemented in certain examples of the disclosed technology.

FIG. 3 illustrates a user wearing a brassiere around their torso, as can be implemented in certain examples of the disclosed technology.

FIGS. 4A-4B are drawings illustrating examples of torso straps, as can be implemented in certain examples of the disclosed technology.

FIG. 5 is a set of drawings shown example of a torso strap been worn by two different users, as can be implemented in certain examples of the disclosed technology.

FIG. 6 is a set of drawings showing examples of a torso strap when worn by two different users, as can be implemented in certain examples of the disclosed technology.

FIGS. 7A-7B are drawings of another example of a torso strap, as can be implemented in certain examples of the disclosed technology.

FIGS. 8A-8B are drawings illustrating examples of torso straps, as can be implemented in certain examples of the disclosed technology.

FIGS. 9A-9B are drawings illustrating examples of torso straps, as can be implemented in certain examples of the disclosed technology.

### DETAILED DESCRIPTION

Disclosed herein are representative embodiments of methods, apparatus, and systems for the design, manufacture, and assembly of torso straps that can be configured to mount a circuit. This disclosure is set forth in the context of representative embodiments that are not intended to be limiting in any way.

The systems, methods, and apparatus described herein should not be construed as being limiting in any way. Instead, this disclosure is directed toward all novel features and aspects of the various disclosed embodiments, alone and in various combinations and subcombinations with one another. The disclosed systems, methods, and apparatus are not limited to any specific aspect or feature or combinations thereof, nor do the disclosed things and methods require that any one or more specific advantages be present or problems be solved. Furthermore, any features or aspects of the disclosed embodiments can be used in various combinations and subcombinations with one another.

Apparatus can be manufactured, provided, and configured having disclosed aspects in combination and sub-combination with one another. Such apparatus can be manufactured or used to perform disclosed methods in combination and sub-combination with one another. One aspect of the disclosed technology that can be implemented in certain example apparatus includes an apparatus including a strap configured to be worn around a torso, the strap including: a narrower portion adapted to encircle the torso. The apparatus also includes a wider portion configured to situate a circuit at least partially offset from the narrower portion of the strap. The apparatus also includes at least one sensor coupled to a first surface of the strap. The apparatus also includes at least one wire being in electrical or optical communication between the circuit and the at least one sensor. Other examples of this aspect include manufacturing such apparatus or using such apparatus to perform disclosed methods.

Certain implementations of the apparatus can also include one or more of the following features. In some examples of the apparatus, the narrower portion defines a first longitudinal axis; the wider portion includes a lobe that extends along a second axis, the wider portion being further situated to situate the circuit along the second axis and at least partially offset from the first axis. In some examples of the apparatus, the circuit is completely offset from the second axis. In some examples of the apparatus, the circuit is completely offset from a region formed by the strap along the first axis. In some examples of the apparatus, the wider portion has a rounded semi-circular shape extending from the narrower portion. In some examples of the apparatus, the wider portion is adapted to avoid an area of a brassiere when the brassiere and the strap are worn around the torso. In some examples of the apparatus, the wider portion is adapted to position the circuit above or below an area of a brassiere when the brassiere and the strap are worn around the torso. In some examples of the apparatus, the wider portion is adapted to position the circuit away from an underwire or other semirigid structure of a brassiere when the strap and the brassiere are worn around the torso. In some examples the apparatus further includes the circuit being in physical and electrical communication with the torso strap, the circuit being situated at least halfway outside the narrower portion of the strap. In some examples of the apparatus, the circuit is situated on a second surface of the strap obverse to the first surface of the strap. In some examples of the apparatus, the circuit includes a processor, the processor being configured to receive signals from the at least one sensor via the at least one wire. In some examples, the apparatus further includes a fastener to connect to at least two ends of the narrower portion of the strap. In some examples, the sensor of the torso strap is a conductive pad.

In some examples of the disclosed technology, a method includes providing disclosed apparatus to be worn about the torso of a user and using the apparatus to monitor at least one health characteristic of the user. In some examples, the at least one health characteristic includes at least one of the following: pulse, heart rate, temperature, blood oxygen, velocity, displacement, attitude, time, or moisture.

In some examples of the disclosed technology, a method of manufacturing an apparatus includes providing a strap configured to be worn around a torso, the strap including: a narrower portion adapted to encircle the torso, and a wider portion including a region to situate a circuit at least partially offset from the narrower portion of the strap. The method further includes attaching a circuit to the region, attaching at least one sensor to or below a first surface of the strap, and connecting at least one wire in electrical communication between the circuit and the at least one sensor.

Although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed things and methods can be used in conjunction with other things and methods. Additionally, the description sometimes uses terms like “produce,” “generate,” “fabricate,” “receive,” “position,” and “situate” to describe the disclosed methods. These terms are high-level descriptions of the actual operations that are performed. The actual operations that correspond to these terms will vary depending on the particular implementation and are readily discernible by one of ordinary skill in the art having the benefit of the present disclosure.

As used in this application and in the claims, the singular forms “a,” “an,” and “the” include the plural forms unless the context clearly dictates otherwise. Additionally, the term “includes” means “comprises.” Moreover, unless the context dictates otherwise, the term “coupled” means mechanically, electrically, or electromagnetically connected or linked and includes both direct connections or direct links and indirect connections or indirect links through one or more intermediate elements not affecting the intended operation of the described system.

Additionally, certain terms may be used such as “up,” “down,” “upper,” “lower,” and the like. These terms are used, where applicable, to provide some clarity of description when dealing with relative relationships. But, these terms are not intended to imply absolute relationships, positions, and/or orientations.

Theories of operation, scientific principles, or other theoretical descriptions presented herein in reference to the apparatus or methods of this disclosure have been provided for the purposes of better understanding and are not intended to be limiting in scope. The apparatus and methods in the appended claims are not limited to those apparatus and methods that function in the manner described by such theories of operation.

Some of the disclosed methods can be implemented using an electronic circuit (e.g., a processor, field programmable gate array, microcontroller, or other suitable electronic device) to collect data from sensors and communicate with other electronic circuits. In some examples, a processor executes computer-executable instructions stored on one or more computer-readable media (e.g., non-transitory computer-readable storage media, such as one or more optical media discs, volatile memory components (such as DRAM or SRAM), or nonvolatile memory components (such as hard drives and solid state drives (SSDs))) and executed on a computer (e.g., any commercially available computer, including smart phones or other mobile devices that include computing hardware). Any of the computer-executable instructions for implementing the disclosed techniques, as well as any data created and used during implementation of the disclosed embodiments, can be stored on one or more computer-readable media (e.g., non-transitory computer-readable storage media). The computer-executable instructions can be part of, for example, a dedicated software application, or a software application that is accessed or downloaded via a web browser or other software application (such as a remote computing application). Such software can be executed, for example, on a single local computer (e.g., as an agent executing on any suitable commercially available computer) or in a network environment (e.g., via the Internet, a wide-area network, a local-area network, a client-server network (such as a cloud computing network), or other such network) using one or more network computers.

For clarity, only certain selected aspects of the software-based implementations are described. Other details that are well known in the art are omitted. For example, it should be understood that the disclosed technology is not limited to any specific computer language or program. For instance, the disclosed technology can be implemented by software written in C, C++, Java, or any other suitable programming language. Likewise, the disclosed technology is not limited to any particular computer or type of hardware. Certain details of suitable computers and hardware are well-known and need not be set forth in detail in this disclosure.

Furthermore, any of the software-based examples (comprising, for example, a circuit mounted on a torso strap accessing computer-executable instructions for causing a computer to perform any of the disclosed computer-implemented methods) can be uploaded, downloaded, or remotely accessed through a suitable communication means. Such suitable communication means include, for example, the Internet, the World Wide Web, an intranet, software applications, cable (including electrical wires and fiber optic cable or “wires”), magnetic communications, electromagnetic communications (including RF, microwave, and infrared communications), electronic communications, or other such communication means.

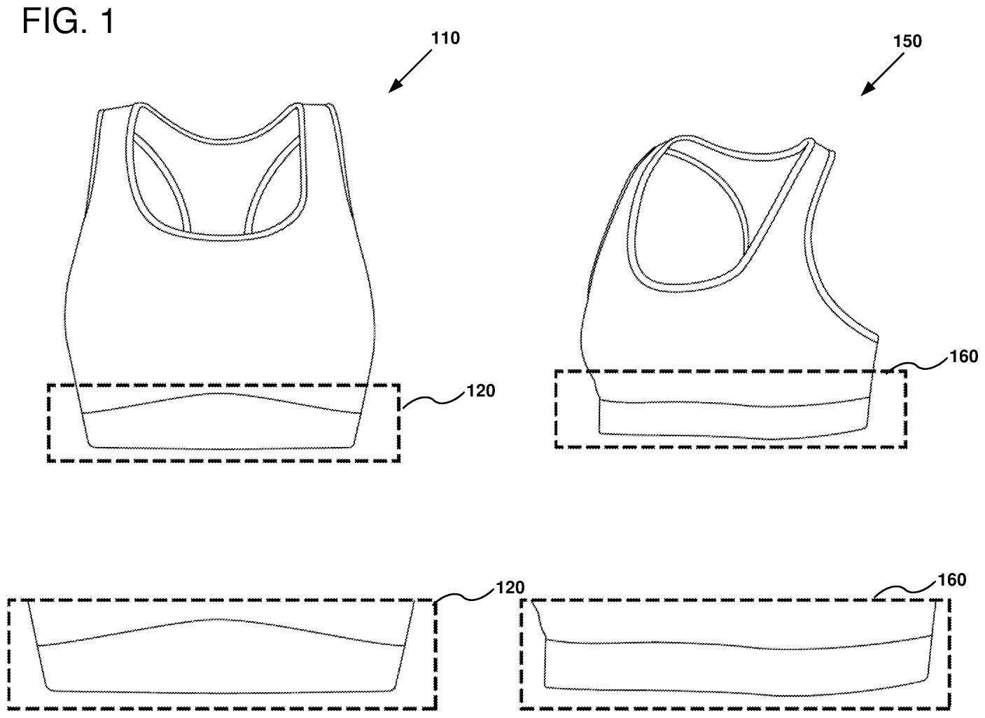

FIG. 1 illustrates two examples of brassieres (which may also be referred to as a “bra”) that can be worn about a user's torso in conjunction with certain examples of the disclosed technology. A first brassiere 110 is shown that does not include an elastic strap or underwire in the region 120 shown. A second brassiere 150 is shown that includes an elastic strap in the region 160 indicated. Other types of bras can include metal or other rigid or semi-rigid materials in the indicated regions. Use of such materials can cause discomfort or otherwise interfere with placement of circuitry in the indicated regions.

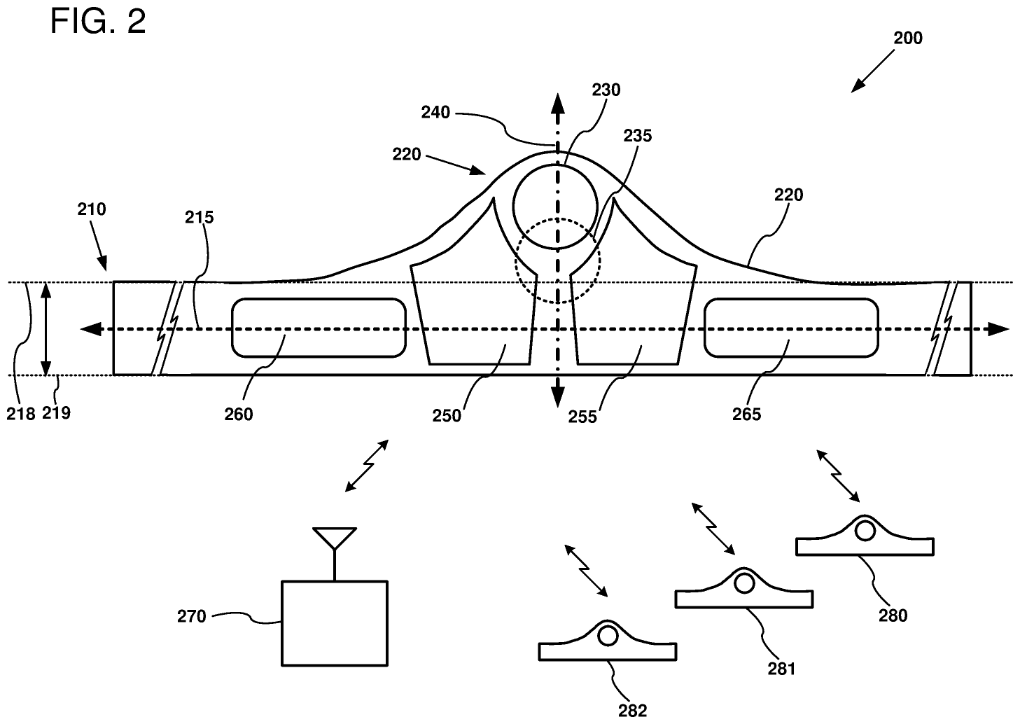

FIG. 2 is a drawing of an apparatus 200 including a strap configured to be worn around a user's torso, as can be implemented in certain examples of the disclosed technology. The strap includes a narrower portion 210 adapted to encircle the torso of the user, the narrower portion defining a first longitudinal axis 215. The width of the narrower portion 210 of the strap is indicated by two dashed lines 218 and 219. The strap also includes a wider portion of 220 configured to situate a circuit 230 at least partially offset from the narrower portion of the strap. In the illustrated example, the circuit 230 is completely offset from the narrower portion of the strap. FIG. 2 also shows an alternate location 235 where the circuit 230 can be placed instead, partially offset from the narrower portion of the strap as indicated by the dashed circle. In the illustrated example, the wider portion 220 includes a lobe that extends a long a second axis 240. Thus, the wider portion of the strap is configured to situate the circuit 230 along the second axis 240 at least partially offset from the first longitudinal axis 215. In the illustrated example, the circuit 230 includes electronics circuitry and a housing in the circular mounting region indicated. Additional wires, cables, printed circuit boards (rigid or flexible), and/or other passive or active electrical components may be included within the mounting region designated in FIG. 2 as the circuit 230.

The apparatus 200 in this example includes optional plates 250 and 255. The plates 250 and 255 are formed from a semi-rigid material that can be used to support the lobe on which the circuit 230 is situated and prevent the lobe from folding away from the torso of the user. The apparatus 200 further includes sensors 260 and 265 that can be configured so that they are in contact with the user's torso. The sensors 260 and 265 can be used to generate electrical or optical signals that can be communicated to the circuit 230. At least one wire being in electrical or optical communication is provided to communicate the signal from the sensors to the circuit. In other examples, the sensor may be in wireless communication with the circuit. In some examples, the sensors 260 and 265 are situated on a first surface of the narrower portion of the strap and the circuit 230 is situated on a second surface of the wider portion of the strap. In some examples, the sensor includes a conductive pad that can be placed in contact with the torso of the user.

The apparatus 200 can be arranged according to a number of different variations. For example, in some examples, the circuit 230 is completely offset from the narrower portion of the strap. In other examples, the circuit is only partially offset from the region formed by the narrower portion of the strap, and some portion of the circuit mounting region overlaps the narrower portion of the strap. In some examples, the wider portion of the strap has a rounded semicircular shape extending from the narrower portion. In some examples, the wider portion of the strap is adapted to avoid an area of a brassiere when the brassiere and the strap are worn around the torso. In some examples, the wider portion is adapted to position the circuit 230 above or below an area of the brassiere when the brassiere and the strap are worn around the torso. In some examples, the wider portion 220 is adapted to position the circuit 230 away from an underwire or other semi-rigid structure of a brassiere when the strap and the brassiere are worn around the torso. In some examples of the apparatus, the circuit 230 is mounted to the circuit mounting region. In some examples of the apparatus, the circuit is mounted at least halfway outside the narrower portion of the strap. In other words, at least half of the area of the circuit is situated outside of the width of the narrower portion of the strap. In some examples, the circuit is situated on a second surface of the strap obverse to the first surface of the strap to which the sensor(s) our mounted. In some examples, the circuit 230 includes a processor, such as a microprocessor, a microcontroller, a soft processor, or other suitable processor that is configured to receive signals from at least one of the sensors 260 and/or 265 via a wire. In some examples, the strap further includes a fastener to connect at least two ends the narrower portion of the strap around the torso of the user.

FIG. 2 further includes a wireless access point 270 and a number of additional torso straps 280, 281, and 282 that have a similar construction as the illustrated torso strap apparatus 200. The wireless access point 270 can communicate with the circuit 230 of the torso strap via wireless communication, for example by using Wi-Fi or a Bluetooth connection. Similarly, the additional torso straps 280, 281, and 282 can also communicate with the circuit 230 via wireless communication. Thus, data about wearers of the respective torso straps can be communicated with other torso straps or with a wireless access point 270. Data collected from the users can be analyzed in order to make determinations regarding user health or performance in sporting events or other such applications.

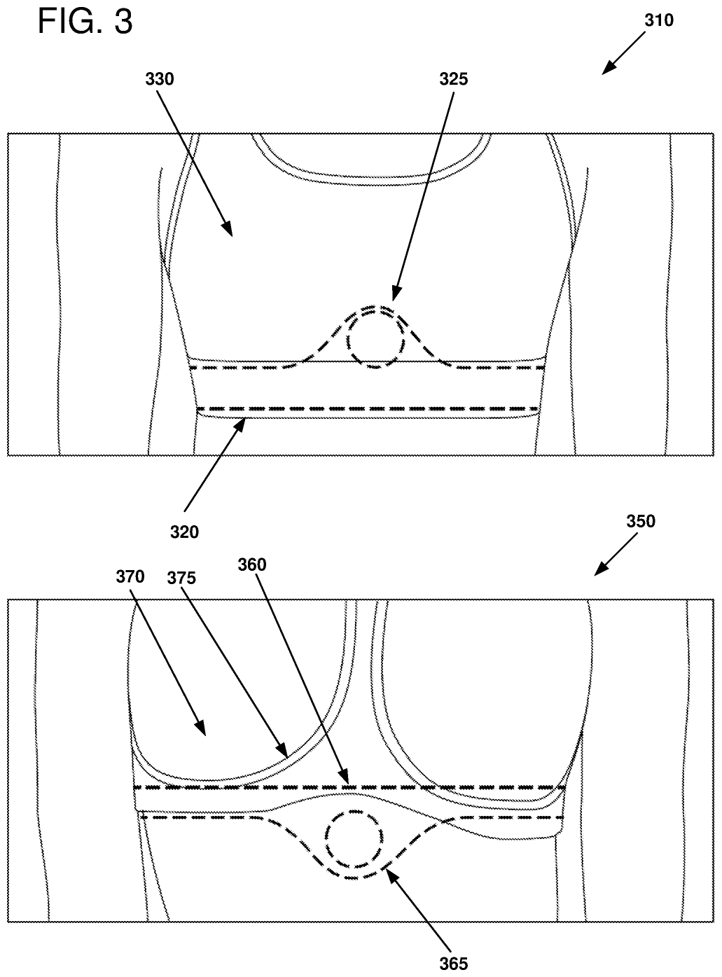

FIG. 3 includes two drawings 310 and 350 of a user wearing a brassiere around their torso and an indication of where a torso strap could be situated around the user's torso, in accordance with certain examples of the disclosed technology. In the first drawing 310, the user is shown wearing a torso strap having a narrower portion 320 that encircles the user's torso. The torso strap further includes a wider portion that is configured to situate a circuit offset from the narrower portion of the strap. As shown in the first drawing 310, the user's brassiere 330 has an elastic strap running parallel to the narrower portion 320 of the torso strap that encircles the user's torso. Because the wider portion 325 is offset from the area where the elastic strap of the brassiere makes contact with the user's torso, the circuit situated in the wider portion 325 does not interfere with the elastic strap of the brassiere 330.

FIG. 3 further includes a second drawing 350 of the user wearing a brassiere 370 having an underwire around their torso. In this example, the torso strap has been inverted so that a narrower portion 360 runs parallel around the user's torso with an underwire 375 of the brassiere 370. In this example, a wider portion 365 of the torso strap situated below the underwire 375 of the brassiere 370 so that the circuit can be situated to avoid interference with the underwire of the brassiere 370.

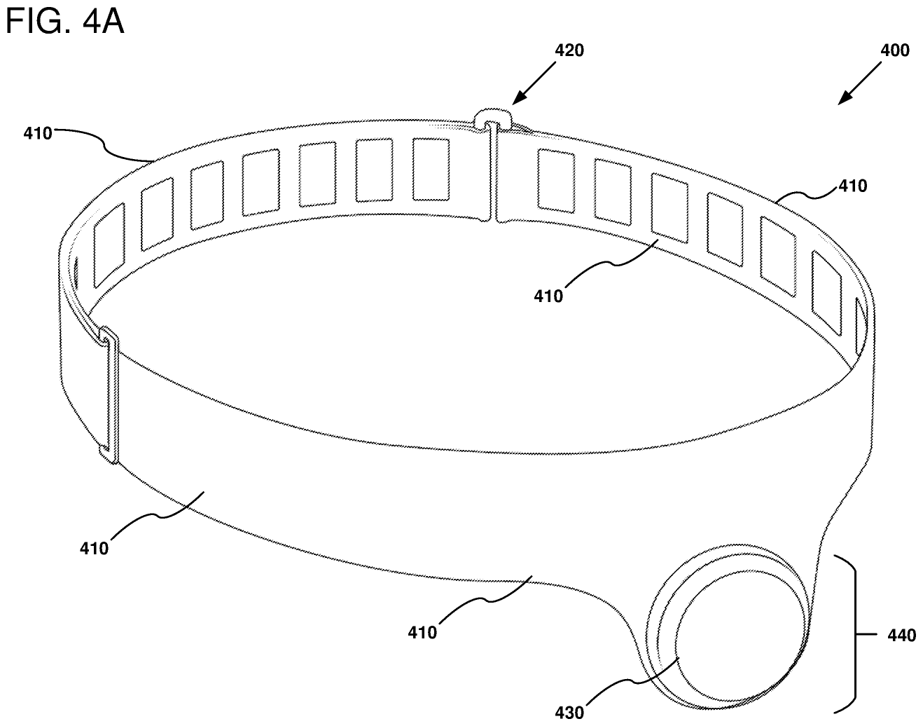

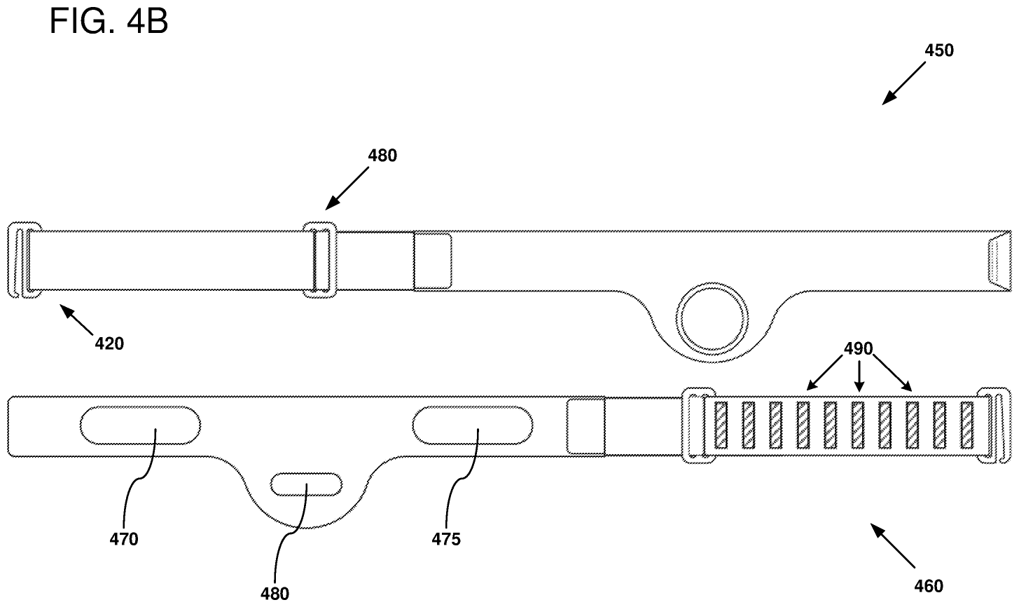

FIG. 4A is a drawing 400 illustrating an example of a torso strap that can be used to situate circuits offset from a narrower portion 410 of the strap, as can be implemented in certain examples of the disclosed technology. In the drawing 400 of FIG. 4A, the torso strap is shown in a three-dimensional representation with two ends of the narrower portion 410 connected. As shown, the strap has a narrower portion 410 having an adjustable length and is configured to use a connector 420 (here, a thin g-hook) to couple two ends of the torso strap. The torso strap further includes a circuit housing 430 that is situated on a surface of a wider portion 440 of the torso strap obverse from the surface that comes in contact with the user's torso. Because the circuit is offset from the narrower portion, physical interference with other objects that may be situated above or below the narrower portion of the strap can be avoided.

FIG. 4B is a set of two drawings 450 and 460 illustrating the torso strap of FIG. 4A when the strap has been laid flat. As shown, the exterior of the torso strap is shown in drawing 450, including the G-hook connector 420 and the circuit housing 430. The strap can be adjusted in length by moving the D-hook 480 shown. The lower drawing 460 shows the interior surface of the torso strap obverse to the exterior surface of the torso strap. As shown, there are three sensor pads 470, 475, and 480. Silicone has been added where indicated (e.g., at 495) to provide additional rigidity to portions of the torso strap.

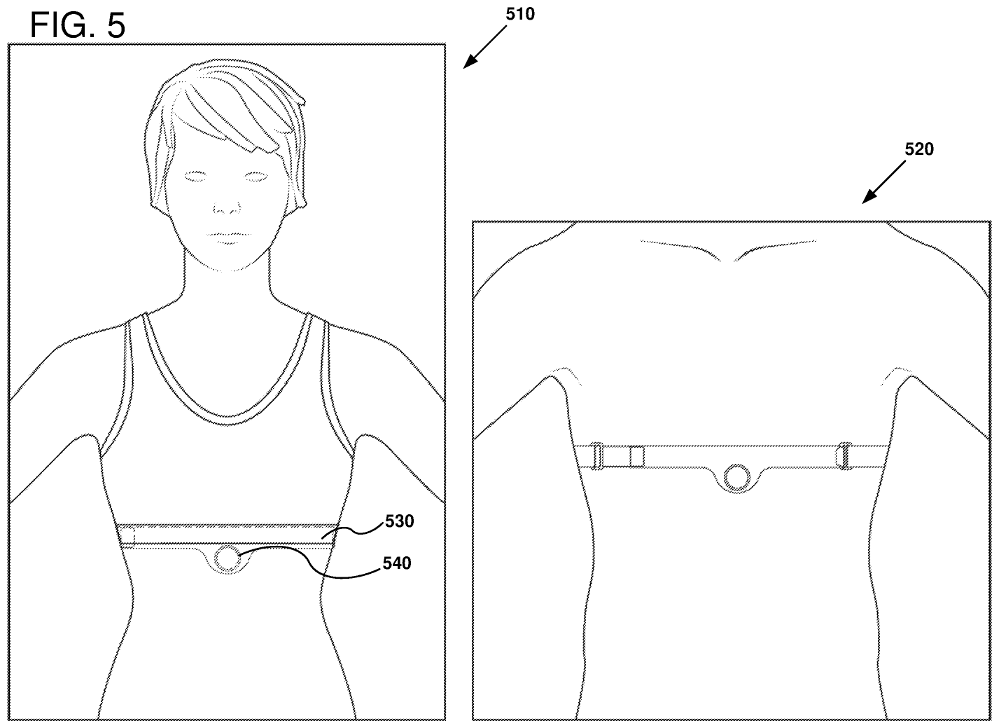

FIG. 5 is a set of drawings showing an example of a torso strap being worn by two different users 510 and 520. The first user is shown wearing a sports brassiere that is in contact with the narrower portion 530 of the torso strap, but a portion of the torso strap adapted to connect to a circuit housing positions the circuit housing 540 out of the way of the sports brassiere, thereby avoiding physically interfering with the sports brassiere. The second user is a male user, who can also wear the same torso strap as the first user as shown.

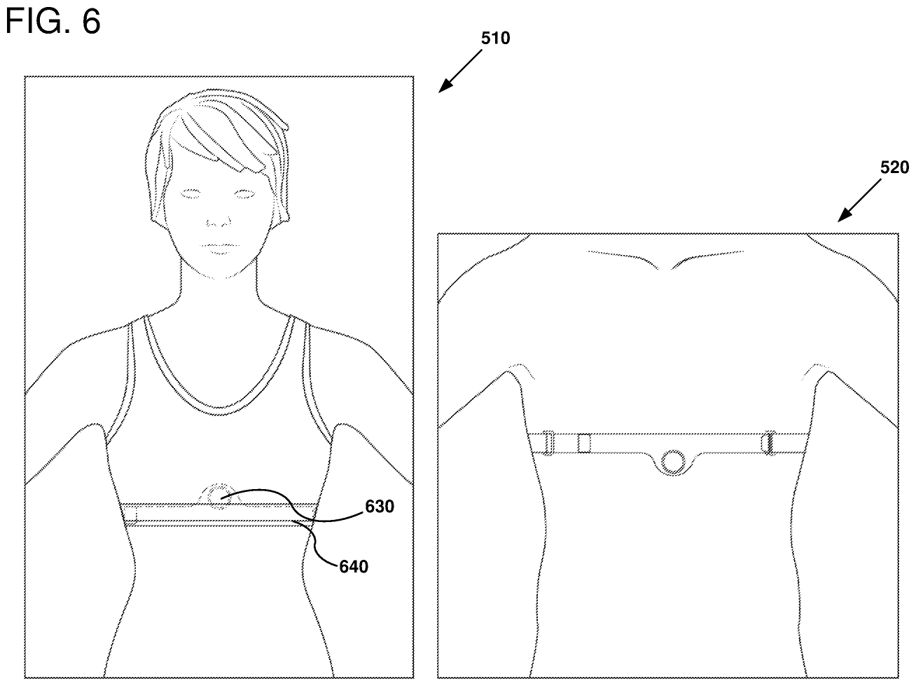

FIG. 6 is a set of drawings shown example of the torso strap of FIG. 5 when worn by the female user 510 in an inverted position. In the drawing of FIG. 6, the torso strap has been flipped such that the circuit's position 630 is above an elastic band 640 in the first user's sports brassiere. The user can select the position of the torso strap based on their personal preferences.

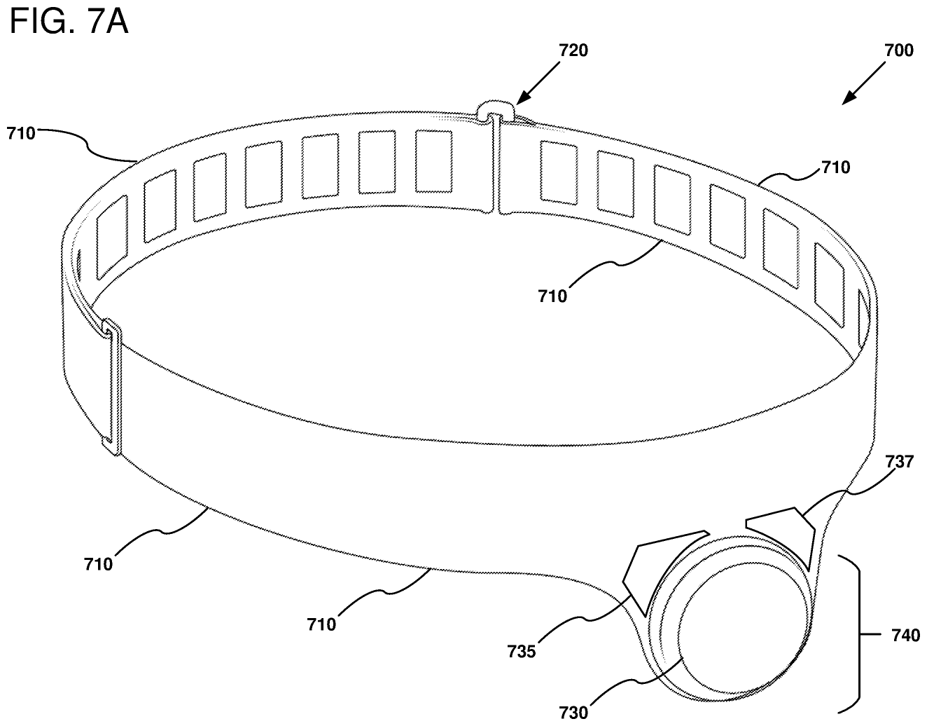

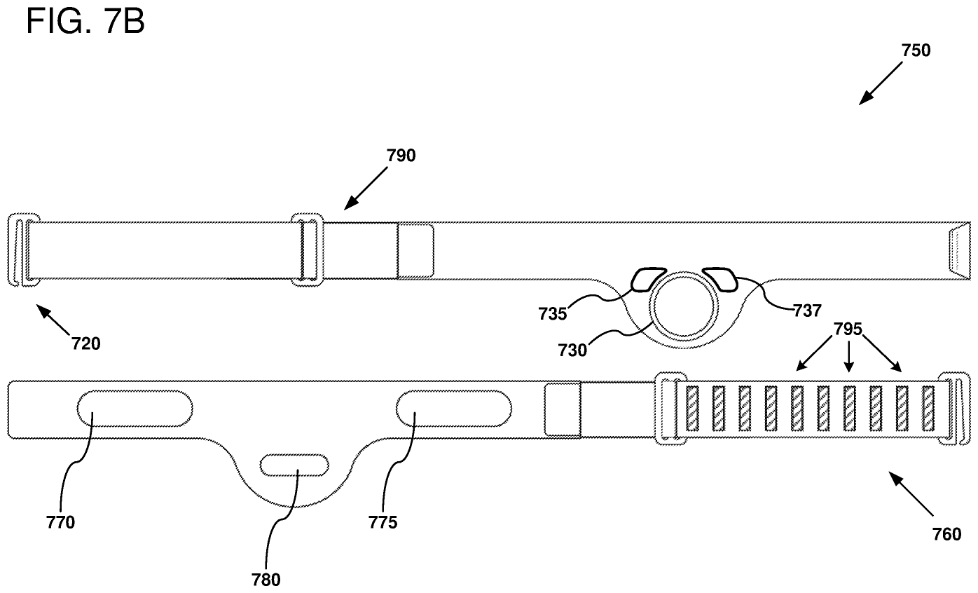

FIGS. 7A and 7B are drawings 700, 750, and 760 of another example of a torso strap, as can be used in certain examples of the disclosed technology. This torso strap is similar to the torso strap of FIGS. 4A-4B in some respects. In the illustrated example, the narrower portion 710 of the torso strap is wider (42 mm) than the narrower portion 310 (30 mm) of the torso strap of FIG. 4A. It includes a G-hook 720 connecting the ends of the narrower portion. In the illustrated example, the circuit housing 730 is offset from a portion but not all of the narrower portion 710 of the strap. Thus, the wider portion 740 is partially offset from the narrower portion of the torso strap. Two raised, padded plates 735 and 737 are formed from a semi-rigid material that can be used to support the lobe on which the circuit housing 730 is situated and prevent the lobe from folding away from the torso of the user.

FIG. 7B includes two drawings 750 and 760 where the torso strap has been laid flat. As shown, the torso strap includes three sensor pads 770, 775, and 780 as well as a D-hook 790 that can be used to adjust the length of the torso strap. Silicone has been added where indicated (e.g., at 795) to provide additional rigidity to portions of the torso strap.

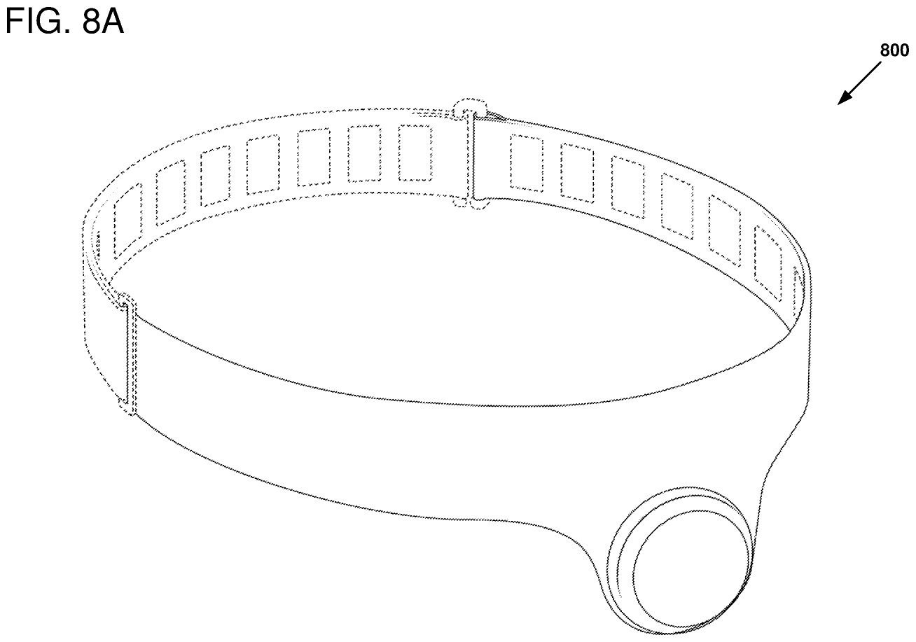

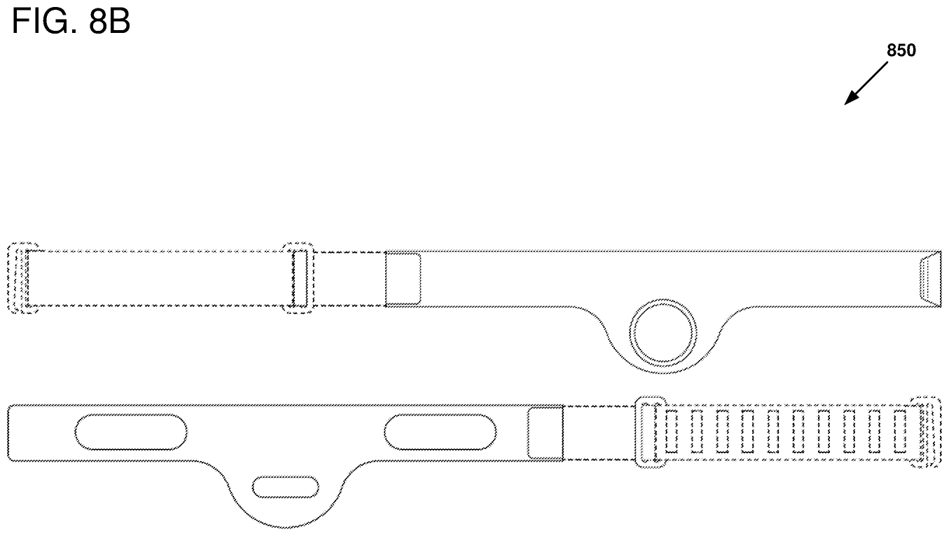

FIGS. 8A and 8B are alternative drawings 800, 850 of the torso strap illustrated in FIGS. 4A and 4B.

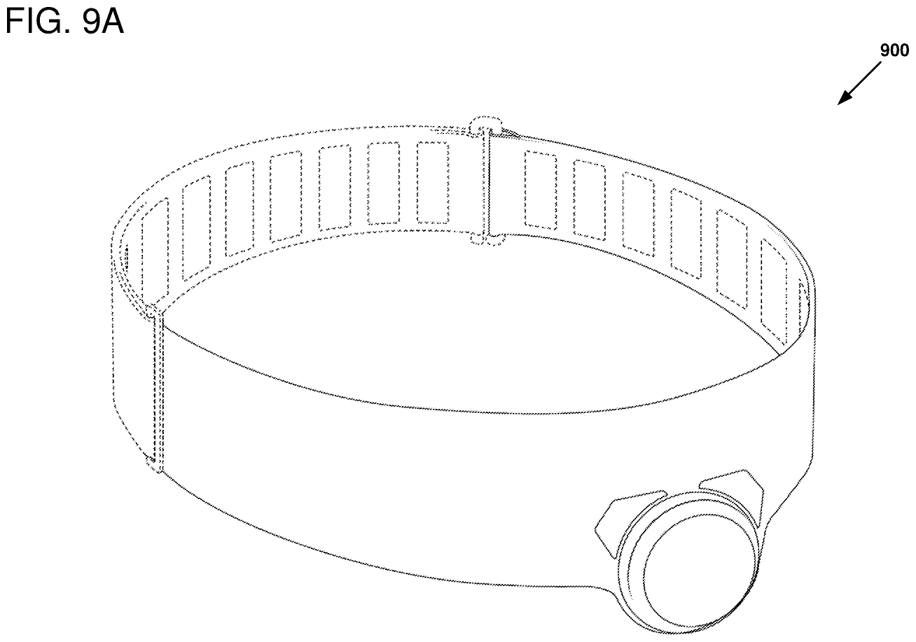

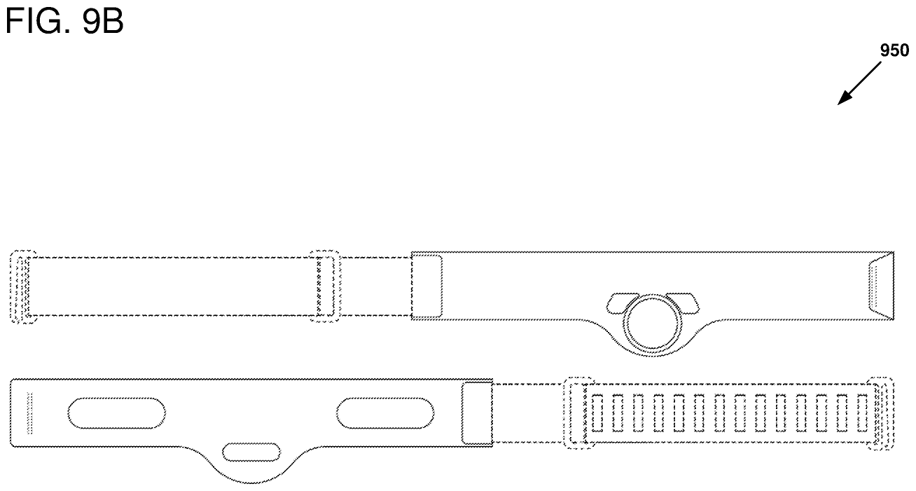

FIGS. 9A and 9B are alternative drawings 900, 950 of the torso strap illustrated in FIGS. 7A and 7B.

In view of the many possible embodiments to which the principles of the disclosed subject matter may be applied, it should be recognized that the illustrated embodiments are only preferred examples and should not be taken as limiting the scope of the claimed subject matter to those preferred examples. Rather, the scope of the claimed subject matter is defined by the following claims. I therefore claim as my invention all that comes within the scope of these claims.

What is claimed is:

1. An apparatus comprising a strap configured to be worn around a torso, the strap comprising:
a narrower portion adapted to encircle the torso;
a wider portion configured to situate a circuit at least partially offset from the narrower portion of the strap;
at least one sensor coupled to a first surface of the strap; and
at least one wire being in electrical or optical communication between the circuit and the at least one sensor.

2. The apparatus of claim 1, wherein:
the narrower portion defines a first longitudinal axis; and
the wider portion comprises a lobe that extends along a second axis, the wider portion being further situated to situate the circuit along the second axis and at least partially offset from the first axis.

3. The apparatus of claim 2, wherein the circuit is completely offset from the second axis.

4. The apparatus of claim 2, wherein the circuit is completely offset from a region formed by the strap along the first axis.

5. The apparatus of claim 1, wherein the wider portion has a rounded semi-circular shape extending from the narrower portion.

6. The apparatus of claim 1, wherein the wider portion is adapted to avoid an area of a brassiere when the brassiere and the strap are worn around the torso.

7. The apparatus of claim 1, wherein the wider portion is adapted to position the circuit above or below an area of a brassiere when the brassiere and the strap are worn around the torso.

8. The apparatus of claim 1, wherein the wider portion is adapted to position the circuit away from an underwire or other semirigid structure of a brassiere when the strap and the brassiere are worn around the torso.

9. The apparatus of claim 1, further comprising the circuit, the circuit being situated at least halfway outside the narrower portion of the strap.

10. The apparatus of claim 1, wherein the circuit is situated on a second surface of the strap obverse to the first surface of the strap.

11. The apparatus of claim 1, wherein the circuit comprises a processor, the processor being configured to receive signals from the at least one sensor via the at least one wire.

12. The apparatus of claim 1, further comprising a fastener to connect to at least two ends of the narrower portion of the strap.

13. The apparatus of claim 1, wherein the sensor is a conductive pad.

14. A method comprising:
providing a strap to be worn about the torso of a user, the strap comprising:
a narrower portion adapted to encircle the torso,
a wider portion having a circuit situated at least partially offset from the narrower portion of the strap,
at least one sensor coupled to a first surface of the strap, and
at least one wire being in electrical or optical communication between the circuit and the at least one sensor; and
with the circuit and the at least one sensor, monitoring at least one health characteristic of the user.

15. The method of claim 14, wherein the at least one health characteristic comprises at least one of the following: pulse, heart rate, temperature, blood oxygen, velocity, displacement, location, position, attitude, time, or moisture.

16. A method of manufacturing an apparatus, the method comprising:
providing a strap configured to be worn around a torso, the strap comprising:
a narrower portion adapted to encircle the torso, and
a wider portion including a region to situate a circuit at least partially offset from the narrower portion of the strap;
attaching a circuit to the region;
attaching at least one sensor to or below a first surface of the strap;
connecting at least one wire in electrical communication between the circuit and the at least one sensor.

17. The method of claim 16, wherein the attaching situates the circuit at least halfway outside the narrower portion of the strap.

18. The method of claim 16, wherein the attaching situates the circuit on a second surface of the strap obverse to the first surface of the strap.

19. The method of claim 16, wherein:
the narrower portion defines a first longitudinal axis; and
the wider portion comprises a lobe that extends along a second axis, the wider portion being further situated to situate the circuit along the second axis and at least partially offset from the first axis.

20. The method of claim 19, wherein the circuit is completely offset from a region formed by the strap along the first axis.
